(19) United States
(12) Patent Application Publication
(10) Pub. No.: US 2013/0066284 A1
(43) Pub. Date: Mar. 14, 2013

(54) Connecting device for use in vacuum therapy of wounds

(71) Applicants: Pierre Croizat, Herbrechtingen (DE); Axel Eckstein, Heidenheim (DE); Julia Wingender, Ueberlingen (DE)

(72) Inventors: Pierre Croizat, Herbrechtingen (DE); Axel Eckstein, Heidenheim (DE); Julia Wingender, Ueberlingen (DE)

(21) Appl. No.: 13/602,338

(22) Filed: Sep. 4, 2012

Related U.S. Application Data
(60) Provisional application No. 61/573,186, filed on Sep. 21, 2011.

(30) Foreign Application Priority Data
Sep. 8, 2011 (DE) 10 2011 082 341.7

(51) Int. Cl. A61M 1/00; A61M 35/00

(52) U.S. Cl. 604/313

(57) ABSTRACT

A connecting device (2) for use in the vacuum therapy of wounds has a suction tube (10) and a vacuum-tight and flexible support means (12) for the suction tube (10), on which the suction tube (10) is held in a vacuum-tight manner, wherein the support means (12) can be applied in an essentially vacuum-tight manner to a vacuum dressing (6) that overlaps the wound and seals it with respect to the atmosphere. A fluid feed tube (40) for conveying a fluid medium to the wound space is located in the same plane as the suction tube (10) and extends to the support means (12). The fluid feed tube (40) extends sealingly through an opening (42) in the support means (12) and projects through an opening (20) in the vacuum dressing (6) into the wound space or into a wound support (50) provided there.

This application claims benefit of 61/573,186 filed Sep. 21, 2011 as well Paris Convention priority of DE 10 2011 082 341.7 filed Sep. 8, 2011 the entire disclosures of which are hereby incorporated by reference.

### BACKGROUND OF THE INVENTION

The invention relates to a connecting device for use in the vacuum therapy of wounds, having a suction tube to which a vacuum can be applied and a two-dimensional vacuum-tight and flexible support means for the suction tube, on which the suction tube is held in a vacuum-tight manner, wherein the support means can be applied in an essentially vacuum-tight manner to a vacuum dressing that overlaps the wound and seals it with respect to the atmosphere, so that the suction tube can communicate with the wound space through at least one opening in the support means and in the vacuum dressing, wherein the suction tube is constituted to be flexible and flat and is, in its intended use, non-detachably and two-dimensionally connected to the support means in a wound-side longitudinal section with at least 70% of its surface projected perpendicularly onto the support means, wherein the support means and the suction tube are mutually parallel with their main planes resulting from their flat shape, and wherein the thickness (D) of the assembly comprising the suction tube and support means is no more than 7 mm.

The importance of the vacuum therapy of wounds, in particular, of wounds that are deep and whose healing is therefore problematical, has recently grown. Vacuum therapy means that a region of the body or wound that would otherwise be exposed to the atmosphere is pressure-tightly and vacuum-tightly sealed against the environment, that is, the atmosphere in which we live and breathe, by means to be described in more detail below, wherein a pressure that is reduced with respect to atmospheric pressure in a manner still be described, which is therefore negative pressure with respect to the atmosphere, can be applied and continuously maintained within the sealed wound space. Where mention is made herein of a vacuum on the region, this refers to a pressure range of typically between 0 and 250 mmHg (mm of mercury) below ambient atmospheric pressure. It has been shown that this is conducive to wound healing. Vacuum-tight sealing can be achieved with a vacuum dressing that, for example, can comprise a pressure-tight or vacuum-tight film layer, which is typically adhesively bonded to an intact region of the body surrounding the wound, thus providing a tight seal. To supply vacuum to the wound space and maintain it there, from a device for producing a vacuum, that is, a vacuum pump in the broadest sense, in the case of the systems stated here for the vacuum therapy of wounds, conduits can be used to which a vacuum is applied that act in conjunction with the vacuum dressing by means of a connecting device to apply a vacuum on or in the wound space.

Such a connecting device of the type stated in the introduction is known from DE 10 2009 060 596 Al.

The object of this invention is to provide a connecting device of the type stated in the introduction, with which more varied use and therapy are possible.

### SUMMARY OF THE INVENTION

This object is inventively achieved with a connecting device of the stated type by providing a fluid feed tube for conveying a fluid medium to the wound space, which is located in the same plane as the suction tube and which extends to the support means, whose thickness is no greater than the thickness of the suction tube, and by leading the fluid feed tube sealingly through an opening in the support means and which protrudes beyond the support means on the side facing the wound and which can project through the opening in the vacuum dressing into the wound space or into a wound support provided there.

The invention suggests provision of an additional fluid feed tube, which projects through the connecting device in such a way that a section of the fluid feed tube is present on the side of the support means facing the wound, which can be used for direct insertion into the wound space or into a wound support provided there in order to implement fluid conveyance directly into the wound space. In this way, the connecting device can be used for vacuum therapy, during which a fluid, in particular, a liquid or a gas, such as, for example, oxygen, in particular, with pharmaceutically effective substances that support wound healing, can be conveyed continuously or discontinuously to the wound space. The connecting device can therefore be used for so-called instillation therapy. The fluid that is fed via the fluid feed tube can also have an antimicrobial or analgesic effect. However, it can also simply be a rinsing solution, such as, in particular, Ringer's solution. Because the fluid feed tube only opens into the wound space beneath the support means, it can be ensured that the liquid to be conveyed actually reaches the wound space and preferably deep into the wound space and, especially preferably, right down to the base of the wound, where it can act in the intended way. The conveyed liquid and, where applicable, any additional wound fluids or wound exudates are removed via the suction tube by known means and suctioned toward a device generating a vacuum. Facilities for generating a vacuum for the vacuum therapy of wounds, which besides having a device for generating a vacuum also have a single-use container to be thrown away preferably after use, for the collection of body fluids, have been described, in particular, in DE 10 2009 038 130 Al and DE 10 2009 038 131 Al of the assignee. Such a facility has a connection for a suction tube leading to the body of the patient as is described here. The features of this facility of the prior art are hereby incorporated by reference. The inventive fluid feed tube and a fluid feed facility with which it is combined can either be achieved independently and separately from a facility for generating a vacuum for medical applications, for example, in the form of an instillation device, or the fluid feed facility can be integrated into the mentioned facility for generating a vacuum.

According to a preferred embodiment of the inventive connecting device, it has proved advantageous if the fluid feed tube extends parallel to the suction tube at least in the region of the connecting device. This simplifies attachment on the vacuum dressing but also the disposition and placement of the conduits around the patient. Irrespective of this, it also proves practical if the conduits extend parallel in the region of the support means.

It will be noted at this point that the two-dimensional vacuum-tight and flexible support means for the suction tube and consequently also for the fluid feed tube is a two-dimensionally constituted interface between the suction tube and the vacuum dressing. The support means together with the suction tube and the additionally brought in fluid feed tube constitutes a flat, flexible, and two-dimensional configuration. Viewed from above, at least, the support means is constituted with more overlap two-dimensionally than the suction tube connected to it and the additionally brought in fluid feed tube. As a result, the support means forms a larger connecting area with the upper surface of the vacuum dressing facing away from the wound.

Furthermore, it is also advantageous if the suction tube and the fluid feed tube are made of a flexible elastomer material with a Shore A hardness of maximum 65, in particular, of 5-65, in particular, of 10-60, in particular, of 15-55. The Shore A hardness is determined according to DIN 53505 dated August 2000, and at 23° C. on a plate-shaped flat and smooth specimen of thickness 6 mm as described in the standard. Correspondingly, it is advantageous if the two-dimensional support means is made of a material that is within the previously mentioned Shore A hardness range.

Further, it proves advantageous if the fluid feed tube exits at an acute angle, in particular, of 10-70°, in particular, of 20-70°from the wound-facing side of the support means. Because of the preferably flexible embodiment of the fluid feed tube, the section of the fluid feed tube that protrudes beyond the underside of the supporting means can easily be brought into the required and desired position relative to the wound space or to the wound support provided there.

According to a further embodiment of the invention, it can be advantageous for certain applications if the fluid feed tube has transverse openings in the wall on an end section protruding from the wound-facing side of the support means.

Even though the length of the section protruding from the wound-facing side of the supporting means is not subject to any manufacturing restrictions, for the majority of applications it is considered advantageous if this length of the protruding section of the fluid feed tube is 0.5-10 cm, in particular, 0.7-5 cm, in particular, 1-3 cm, wherein this length is measured in a longitudinal center axis of the fluid feed tube. In particular, it is conceivable if a relatively long length is provided by the manufacturer, which can be shortened to the required dimension when the connecting device is being attached.

Furthermore, the fluid feed tube can be constituted as a single lumen or double lumen tube, depending on whether it is desirable to convey different fluids.

In a further embodiment of the invention, it is suggested that the fluid feed tube has a disconnection facility in the region between the connecting device and a facility for generating a vacuum, which preferably can be operated manually and which, in particular, can be constituted by a simple clamping means, such as clamping scissors. In this way, fluid can be prevented from dripping out when a dressing is changed when connecting to or disconnecting from a fluid feed facility.

It can furthermore be advantageous if an additional venting tube is provided parallel to the suction tube and if the two tubes communicate with each other at each of their ends in the region of the support means. Such a venting tube serves on the one hand to dissolve blockages in the suction tube by providing an adequate quantity of flowing medium by venting with a gas, preferably air, which in combination with the device generating the vacuum can dissolve a blockage. On the other hand, a venting tube can also be used for the precise vacuum control when, for instance, the vacuum has to be reduced (i.e. the absolute prevailing pressure has to be increased) according to a control program. It is also conceivable for the suction tube, the fluid feed tube, and the venting tube to be integrated in a joint conduit, that is, in particular, by separate lumens of the same, in particular, extruded conduit, wherein, then preferably, the suction tube is constituted between the two other tubes.

Several possibilities are conceivable for attaching the connecting device to the side, facing away from the wound, of a vacuum dressing overlapping the wound: According to one embodiment, the connecting device can be attached to the vacuum dressing by means of an adhesive-coated film layer that either completely or partially overlaps the connecting device. This film layer then extends beyond the two-dimensional extension of the support means. According to a further embodiment, an adhesion layer can be constituted on the wound-facing side of the support means, which in its simplest form can be made of a two-dimensional continuous or interrupted or frame-like adhesive layer. A further embodiment of this variant comprises an at least three-layer adhesion layer on the wound-facing side of the support means, which comprises a middle support layer, a first adhesive layer held on the support layer and facing the support means and a second adhesive layer held on the support layer and facing away from the support means and is constituted such that it does not block at least one opening in the support means.

This inventive embodiment of the connecting device is particularly advantageous because the adhesion layer can typically be fixed to an ideally even extension with the even wound-facing side of the two-dimensional support means, whereas otherwise an additional adhesive film must surround the support means and/or the configuration of the support means, thus adding an additional third dimension, which in practical handling makes the proper procedure of applying and sticking the connecting device to the upper surface, facing away from the wound, of the wound support more difficult because folds are formed. Another advantage of this embodiment derives from the fact that typically different materials are used for the two-dimensional support means on the one hand and the wound dressing on the other hand, for example, silicone, on the one hand and, for example, polyurethane, on the other hand, so that the adhesive agent of an adhesive film cannot be optimally matched to both materials. Thus it is advantageous if the first adhesive layer and the second adhesive layer are formed from different adhesive materials with different adhesive properties, so that they can be selected based on the specific materials of the support means and vacuum dressing.

In particular it can advantageous if the support means is made of silicone and the first adhesive layer comprises a silicone adhesive, at least as the main component. Furthermore, it can prove advantageous if the second adhesive layer comprises an acrylate adhesive, at least as the main component. Acrylate adhesives provide excellent adhesion to polyurethane surfaces. In that case, optimum adhesion can be achieved on a wound dressing with a polyurethane surface.

According to a further inventive idea it is advantageous if the adhesive surface of the first adhesive surface layer facing the support means and/or the adhesive surface of the second adhesive layer facing the vacuum dressing is smaller than 100 cm², in particular, smaller than 50 cm², in particular, smaller than 40 cm², in particular, smaller than 30 cm². It has been shown that in the case of an adhesive material that is optimally matched to the adhesive counterpart, that is, the material of the support means or of the wound dressing, a very much smaller adhesive surface on the connecting device is sufficient to achieve the same adhesive effect. The surface sizes specified above herein refer to the surface projecting from one side of the contact surface in question, that is, quite simply to the size of the adhesive even surface of the first or of the second adhesive layer.

It proves especially advantageous if, in the case of the inventive connecting device, an adhesion layer is used whose first adhesive layer is constituted such that the adhesion layer exhibits a peel adhesion provided by this first adhesive layer with respect to a steel plate of 25 to 40 N/100 mm (100 mm width of the first adhesive layer) and/or its second adhesive layer is constituted such that the adhesion layer exhibits a peel adhesion provided by this second adhesive layer with respect to a steel plate of 70 to 85 N/100 (100 mm width of the second adhesive layer). Herein, the adhesion layer is tested on its own merits, that is, not in combination with the support means. This peel adhesion is defined according to ASTM test method D 3330 M, wherein the dwell times are 1 min, the peel-off angle is 180° (peel-off orientation), and the peel-off speed is 300 mm/min. The test is performed at 22° C. and 50% relative air humidity. These values can be advantageously achieved when the first adhesive layer comprises a silicone adhesive and the second adhesive layer comprises an acrylate adhesive.

It proves advantageous for manufacturing purposes if the first and second adhesive layer have the same two-dimensional extension when projected onto their extension plane, that is, their projections cover the exactly the same area, and, in particular, have the same extension as the support layer. In the latter case, all layers are therefore constituted to cover an identical area. This embodiment proves especially advantageous because in such a case the adhesion layer can be manufactured from an endless flat material strip coated on both sides with adhesive material, where the sections required for the adhesion layer merely have to be cut to length or punched out.

The adhesion layer can be manufactured in an especially advantageous way from a double-sided adhesive strip, wherein one section of the double-sided adhesive strip is separated and configured to the required size and shape to be then applied to the wound-facing side of the support means.

The first adhesive layer of the adhesion layer is advantageously directly applied to the wound-facing side of the support means.

Further it proves advantageous if the first and the second adhesive layer have a thickness of 20 to 400 μm.

The middle support layer can advantageously be a non-woven material, a flat material with a textile binding, such as knitted or woven material, or a plastic film, a metal foil, or a composite material.

It further proves advantageous if the adhesion layer extends over the entire wound-facing side of the support means, wherein here the at least one opening in the support means is left uncovered for vacuum communication purposes.

It is also conceivable and advantageous if the adhesion layer extends in the shape of a frame around the at least one opening in the support means. In this case, the two-dimensional extension of the adhesion layer can also be considerably smaller than the two-dimensional extension of the wound-facing side of the support means. However, it also proves advantageous if the two-dimensional extension of the adhesion layer amounts to at least 50% of the surface of the wound-facing side of the support means.

If further proves advantageous if the second adhesive layer is surrounded by a removable protective layer, which is preferably constituted in two parts, and preferably with a handling tongue and/or a grip area that extends beyond the second adhesive layer.

The width extension of the flat suction tube preferably measures at least 10 mm, in particular, at least 15 mm, and further, at least 18 mm, and, in particular, no more than 30 mm and further especially no more than 25 mm. According to a preferred embodiment of the invention, the suction pipe can be made of a silicone-based material.

Further, it proves advantageous if the inside of the suction tube has integrally molded means, in particular, forming one piece with the material of the suction tube to prevent the suction tube from collapsing when a vacuum is applied. These means for preventing collapse can be constituted by ribs or projections. In a further embodiment of this inventive idea, it is advantageous if the latter extend right through. The suction tube can then advantageously be constituted as an extruded part.

Furthermore, it can prove advantageous if the suction tube comprises multiple pressure-tight channels separated from one another, wherein the suction tube in that case, too, is preferably constituted as one piece, that is, not as a combination of multiple separate channel-forming means. One or more channels can then constitute the fluid feed tube. Another can constitute the venting tube.

The preferably flat suction tube advantageously extends across a certain distance in the longitudinal direction and can then change via a transition or coupling element, not shown in the illustration, which can constitute a plug connection or an adhesive connection, into a common more torsionally rigid round hose, which results in a facility that generates a vacuum, which can be constituted as a stationary appliance or as a mobile device that can be carried on the body of the patient. The transition or coupling element can also be constituted to couple a multiple channel conduit with a multiple channel round hose. A distance of 10-60 cm has proven an advantageous longitudinal extension of the suction tube.

The two-dimensional support means advantageously has a thickness of 0.75-3 mm, in particular, of 1-3 mm. According to a preferred embodiment of the invention, it is made of a silicone-based material. According to a further preferred embodiment, the suction tube and the support means are made of the same elastomer material.

It proves advantageous if the two-dimensional extension of the support means is at least 1.5 times and preferably at least twice the area of the suction tube projected perpendicularly onto the support means because, in this way, the forces introduced through the suction tube on contact are distributed over a larger area and also flexural torques that are exerted on the suction tube are either not transmitted to the vacuum dressing at all or only slightly; they are better absorbed by the plate-shaped support means. It proves sufficient if the area ratio stated above is no more than 5, in particular, no more than 4, wherein the ratio of 2 to 3 has proven advantageous.

The vacuum-tight connection of the flat suction tube with the upper side of the support means facing away from the wound is of functionally essential importance. For this, an adhesive connection using an adhesion promoter in the widest sense can basically be used. Furthermore, a thermal jointing connection, which can correspond to a type of vulcanizing connection, proves advantageous. For example, the previously separately manufactured conduit can be applied to the recently freshly cast and only partially set two-dimensional support means, so that in this way an internal material bond of the two components is achieved without using an additional adhesion promoter.

It proves advantageous if the intercommunicating openings in the suction tube and in the support means cover each other, that is, are aligned with each other. This can be most easily achieved if these openings are constituted simultaneously in both components only after the vacuum-tight connection of the flat conduit has been connected to the two-dimensional support means. This can be achieved, for example, by means of a material forming punching process.

As regards the number and size of the openings in the suction tube and in the two-dimensional support means, it is possible to consider providing a single opening only. However it proves advantageous if multiple openings, in particular, at least two, in particular, at least four openings each cm long are constituted along the suction tube in the vacuum-tight connection area between the suction tube and support means.

It also proves advantageous if the clear opening surface of the openings amounts to 5-50% of the surface of the permanently joined flat sides of the suction pipe and of the support means.

Besides the connecting device as such, the present invention also refers to an aggregate comprising an inventive connecting device, a vacuum dressing, and a wound support on the wound-facing side of the vacuum dressing, wherein the wound support constitutes a through-hole that extends in the direction of the wound depth, into which the fluid feed tube can be inserted by its section protruding from the wound-facing side of the support means. In a further embodiment of this inventive idea, the through-hole in the wound support can be delimited by an insert sleeve. The wound support itself preferably comprises a flexible and compliant foam material.

By means of these further characteristics of the wound support, a simple and reliable application of the fluid feed tube that protrudes into the wound space can be implemented. The fluid feed tube can thus be oriented within the wound space, and thus ensure that the liquids to be conveyed reach the wound base, where they are typically required. In particular, said sleeve prevents introduced liquids from being transferred to the wound support and suctioned out again immediately. Because the fluid feed tube projects into the wound support and preferably into the sleeve provided in it, direct contact of the fluid tube with the wound base and thus irritation of the wound base is reliably avoided.

Further characteristics, details, and advantages can be derived from the appended claims and from the following description of a preferred embodiment of the inventive connecting device and its use in the vacuum therapy of wounds.

The drawing shows:

### BRIEF DESCRIPTION OF THE DRAWING

FIG. 1 a perspective view not true to scale of an inventive connecting device for use in the vacuum therapy of wounds, as well as an indicated wound with a vacuum dressing; and

FIG. 2 a schematic sectional view, not true to scale, of the connecting device according to FIG. 1 with a sectional plane II-II;

FIG. 3 a schematic sectional view not true to scale of the connecting device according to FIG. 1 with sectional plane III-III;

FIG. 4 a schematic sectional view not true to scale of a connecting device in combined operation with a wound support.

### DESCRIPTION OF THE PREFERRED EMBODIMENT

The figures show different views of an inventive connecting device collectively designated with the reference sign 2 for use in the vacuum treatment of wounds. The illustrated connecting device 2 is applied to an upper side 4, facing away from the wound, of a schematic vacuum dressing 6, which overlaps a wound 8 to be treated and which seals vacuum tight with respect to the atmosphere and is fixed by adhesive means in a way still to be described.

The connecting device 2 comprises a flat conduit shown as an example in the illustration, in the shape of a suction tube 10 made of an elastomer flexible material and a two-dimensionally extending, plate-shaped support means 12, which holds and supports the flat suction tube 10, so that the pressure and torsional forces applied to the suction tube 10 are introduced evenly into the large-area support means 12 and can be taken up by the latter.

The suction tube 10 is sufficiently flat that in a longitudinal end section 14, almost 100% of its surface that is projected onto the support means 12 is connected to the support means 12. The relevant flat side 15 of the suction tube 10 can be adhesively fixed or vulcanized, that is, thermally joined, onto the upper side 16, facing away from the wound, of the support means 12.

Preferably, after vacuum-tight joining of the suction pipe 10 with support means 12, openings 18 that pass through the support means 12 and through the suction pipe 10 are formed, which communicate with a schematically shown opening 20 or multiple openings 20 in the vacuum dressing 6 when the connecting device 2 is applied to the vacuum dressing 6, in order to apply a vacuum to the wound space.

The extension of the thickness D of the combination of the suction pipe 10 and support means 12 is no more than 7 mm, preferably, no more than 5 mm, and further, preferably only 3 to 4 mm.

Furthermore, the figures also make clear that the connecting device 2 has an adhesion layer 24 on the wound-facing side 22 of the supporting means 12. By means of this adhesion layer 24, the connecting device 2 can be adhesively fixed to the upper side 4, facing away from the wound, of the vacuum dressing 6. The adhesion layer 24 is constituted as three layers and comprises a middle support layer 26, a first adhesive layer 28 held on the support layer 26 and facing the support means 12 and a second adhesive layer 30 held on the support layer 26 facing away from the support means, that is, facing the vacuum dressing 6. In the preferred case shown in the example, all three layers of the adhesion layer 24 are constituted such that they are coextensive. The adhesion layer 24 is in its entirety cut in lengths and/or punched out from a flat material coated on both sides with adhesive material. In the illustrated example, it is frame shaped, that is, it extends continuously around the circumference and extends around the central section 32 of the support means 12 into which the schematically illustrated openings 18 of the support means 12 and/or the suction pipe 10 open in the direction of the vacuum dressing 6. The adhesive surface of the first and the second adhesive layers 28, 30 in the illustrated example is only approx. 30 cm² in size. However, it is also expressly mentioned at this stage that the adhesion layer 24 can also extend across the entire wound-facing side 22 of the support means 12, wherein accordingly, the openings 18 in the support means 12 and/or in the suction pipe 10 must exhibit aligned or communicating through-openings. The adhesive surface is then only approx. 40 cm².

The adhesive layers 28 and 30 are made of varying adhesive materials, which are correspondingly optimized to the materials of the support means 12 and/or of the vacuum dressing 6. If, for example, the support means 12 is made of silicone, it is recommended that the first adhesive layer 28 comprise a silicone adhesive. If the vacuum dressing 6 for which the connecting device 2 is conceived is made of polyurethane and/or is constituted with an upper side 4, facing away from the wound, made of polyurethane, it is recommended that the second adhesive layer 30 comprise an acrylate adhesive which adheres particularly well to polyurethane. By using the inventive adhesion layer 24 it is quite possible to match both these adhesive layers 28 and 30 optimally to the materials of the support means 12 and/or of the vacuum dressing 6 with which they are adhesively combined and manufactured in this combination.

The second adhesive layer 30 is further surrounded by a two-part removable protective layer, which has a handling tongue 36 for removing both parts.

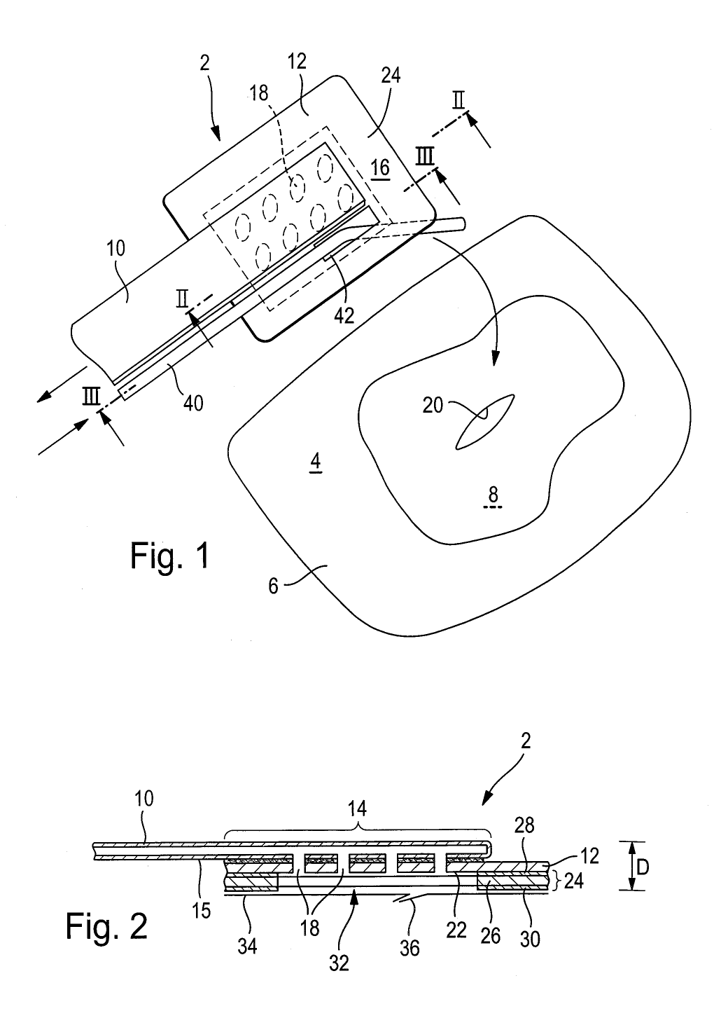

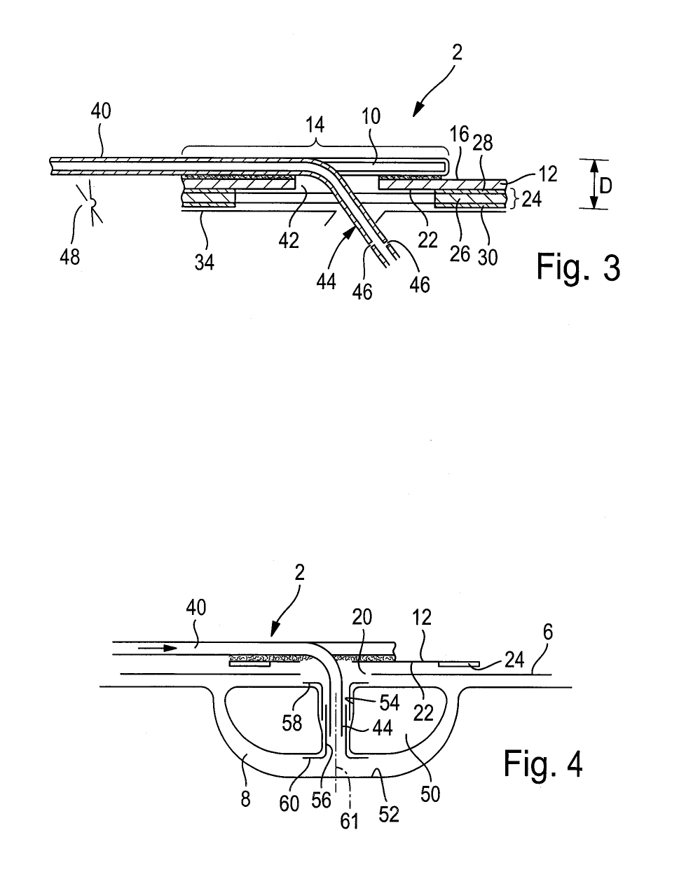

FIGS. 1 and 3 further show a fluid feed tube 40 with which a fluid can be introduced to the wound space, for example, to rinse or instill the wound. The fluid feed tube 40 is preferably conveyed to the support means 12 in the same plane as the suction tube 10 and parallel with the suction tube 10; it is also held by the supporting means 12 and is permanently joined to the upper side 16, facing away from the wound, of the supporting means 12, for example, using adhesive or by material bonding. Inventively, the fluid feed tube 40 extends through a further opening 42 in the support means 12 toward the wound-facing side 22 of the support means 12 and there protrudes at an acute angle toward the wound. Because the fluid feed tube 40 is made of a flexible material in the way described in the introduction, a protruding section 44 of the fluid feed tube 40 can bend easily and essentially without resistance in another desired direction and be oriented with respect to the wound and/or the wound support in a way yet to be described. Additional transverse openings 46, which are merely schematically shown, can be constituted in the wall of the exposed section 44 of the fluid feed tube 40 for specific applications. Furthermore, a disconnection device 48 is schematically shown with which the cross-section of the fluid feed tube 40 can be reduced to zero.

FIG. 4 very schematically shows the application of the inventive connecting device 2 together with a wound support 50 on a wound 8, which is sealed with a vacuum dressing 6, leaving only opening 20 free. In the region of this opening 20 in vacuum dressing 6, the connecting device is applied sealingly, in particular, by means of said adhesion layer 24. Alternatively, the connecting device 2 could be fixed by additional adhesive film that overlaps the connecting device 2 or by sealing the same at the upper side 16, facing away from the wound, of the support means 12. The schematic cross-section of FIG. 4 shows only the sectional plane through the fluid feed tube 40. It shows how the section 44 of the fluid feed tube 40 protruding from the wound-facing side 22 of the support means 12 is introduced into the wound support 50 in the direction of the wound base 52. Inventively, a through-hole 54 is constituted for this in the wound support 50. A sleeve 56 is disposed in the through-hole 54. The sleeve 56 is constituted as two sleeve parts 58, 60, that are inserted through the through-hole from the opposing sides of the wound support 50. The sleeve parts 58, 60 extend in a telescopic manner toward each other along a sleeve longitudinal axis 61. In this way, the sleeve length can be set automatically according to the deformation of the wound support 50. Depending on the vacuum and compression of a wound support 50, preferably formed from absorbent foam, the sleeve length can be set automatically. The sleeve 58 guides the section 44 of the fluid feed tube 40 and prevents the latter from resting against the wound base 52. In this way, liquid can be conveyed via the fluid feed tube 40 to deep regions of the wound 8, preferably down to the wound base 52. The conveyed liquid can then be removed via the suction tube 10 not illustrated in FIG. 4.

The arrangement and embodiment of sleeve 56 is provided purely as an example. It would be equally possible to plug in or insert the section 44 of the fluid feed tube 40 that extends into the wound space into any aperture, cavity, or fold of a wound support.

We claim:

1. A connecting device for use in the vacuum therapy of wounds, the connecting device cooperating with a vacuum dressing that overlaps the wound and seals the wound with respect to atmosphere, wherein the vacuum dressing has an opening, the device comprising:
a flexible and flat suction tube to which a vacuum can be applied;
a two-dimensional vacuum-tight and flexible support means having at least one opening, said support means structured for cooperation with the vacuum dressing in an essentially vacuum-tight manner, said support means holding said suction tube in a vacuum-tight manner, wherein said suction tube passes through said support means opening and through the vacuum dressing opening to communicate with a wound space, said suction tube being non-detachably and two-dimensionally connected to said support means in a wound-side longitudinal section with at least 70% of a surface thereof projected perpendicularly onto said support means, wherein said support means and said suction tube are mutually parallel with main planes thereof resulting from flat shapes thereof, said suction tube and said support means thereby having a combined thickness at most 7 mm; and
a fluid feed tube for conveying a fluid medium to the wound space, said fluid feed tube located in a same plane as said suction tube and extending to said support means, wherein said fluid feed tube has a thickness which is no greater than a thickness of said suction tube, said fluid feed tube extending sealingly through an opening in said support means to protrude beyond a wound-facing side of said support means and project through the opening in the vacuum dressing into the wound space or into a wound support provided there.

2. The connecting device of claim 1, wherein, at least in a region of the connecting device, said fluid feed tube is conveyed parallel to said suction tube.

3. The connecting device of claim 1, wherein said suction tube and said fluid feed tube are made of a flexible elastomer material having a Shore A hardness of no more than 65.

4. The connecting device of claim 1, wherein said fluid feed tube exits from a wound-facing side of said support means at an acute angle or at an angle of 30-70°.

5. The connecting device of claim 1, wherein said fluid feed tube has transverse openings in a wall on an end section protruding from the wound-facing side of said support means.

6. The connecting device of claim 1, wherein a length of a section of said fluid feed tube protruding from a wound-facing side of said support means is 0.5-10 cm, 0.7-5 cm or 1-3 cm.

7. The connecting device of claim 1, wherein said fluid feed tube has a disconnection device in a region between the connecting device and a vacuum generating device.

8. The connecting device of claim 1, further comprising an additional venting tube disposed parallel to said suction tube, said venting tube and said suction tube communicating with each other at respective ends thereof in a region of said support means.

9. The connecting device of claim 1, further comprising an at least three-layer adhesion layer disposed on a wound-facing side of said support means, said adhesion layer comprising a middle support layer, a first adhesive layer held on said support layer and facing said support means, and a second adhesive layer held on said support layer and facing away from said support means, wherein said adhesive layer does not block said at least one opening in said support means.

10. The connecting device of claim 9, wherein said first adhesive layer and said second adhesive layer are made from different adhesive materials with different adhesive properties.

11. The connecting device of claim 9, wherein said support means is made of silicone and said first adhesive layer comprises a silicone adhesive.

12. The connecting device of claim 10, wherein said second adhesive layer comprises an acrylate adhesive.

13. The connecting device of claim 9, wherein an adhesive surface of said first adhesive layer facing said support means and/or an adhesive surface of said second adhesive layer facing the vacuum dressing is smaller than 100 cm², smaller than 50 cm², smaller than 40 cm² or smaller than 30 cm².

14. The connecting device of claim 9, wherein said first adhesive layer is structured such that said adhesion layer exhibits a peel adhesion provided by said first adhesive layer with respect to a steel plate of 25 to 40 N/100 mm or of 27-35 N/100 mm and/or said second adhesive layer is structured such that said adhesion layer has a peel adhesion with respect to a steel plate provided by said second adhesive layer of 70 to 85 N/100 mm or of 70-80 N/100 mm.

15. The connecting device of claim 1, wherein said first and said second adhesive layers exhibit a same two-dimensional extension when projected onto extension planes thereof as well as a same extension as said support layer.

16. The connecting device of claim 1, wherein each of said first and said second adhesive layers has a thickness of 20-400 μm.

17. The connecting device of claim 9, wherein said middle support layer comprises or is made from a non-woven material, a flat material with textile binding, a knitted or woven material, a plastic film, a metal foil or a composite material.

18. The connecting device of claim 9, wherein said adhesion layer extends across an entire wound-facing side of said support means.

19. The connecting device of claim 9, wherein said adhesion layer extends in a shape of a frame around said at least one opening in said support means.

20. The connecting device of claim 9, wherein a two-dimensional extension of said adhesion layer is at least 50% of a surface of a wound-facing side of said support means.

21. The connecting device of claim 9, wherein said second adhesive layer is overlapped by a removable protective layer, which is constituted in two parts and has a handling tongue and/or a grip area that extends beyond said second adhesive layer.

22. A connecting system comprising the connecting device of claim 1, the system further comprising the vacuum dressing and a wound support or a wound support having a foam material on a wound-facing side of said vacuum dressing, wherein said wound support has a through-hole that extends in a direction of a wound depth and into which said fluid feed tube is inserted by a section thereof protruding from a wound-facing side of said support means.

23. The system of claim 22, wherein said through-hole in said wound support is delimited by an insert sleeve.
